(19) United States
(12) Patent Application Publication
(10) Pub. No.: US 2005/0009748 A1
(43) Pub. Date: Jan. 13, 2005

(54) Compositions for delivering peptide YY and PYY agonists

(75) Inventors: Steve Dinh, Ossining, NY (US); Huaizhen Wang, Chappaqua, NY (US); M. I. Gomez-Orellana, New Rochelle, NY (US)

Correspondence Address: DARBY & DARBY P.C., P. O. BOX 5257, NEW YORK, NY, 10150-5257, US

(73) Assignee: Emisphere Technologies, Inc., Tarrytown, NY (US)

(21) Appl. No.: 10/846,954

(22) Filed: May 14, 2004

Related U.S. Application Data
(60) Provisional application No. 60/470,905, filed on May 14, 2003.
(60) Provisional application No. 60/471,114, filed on May 15, 2003.
(60) Provisional application No. 60/506,702, filed on Sep. 25, 2003.
(60) Provisional application No. 60/536,697, filed on Jan. 14, 2004.

(51) Int. Cl. A61K 38/17; A61K 31/195

(52) U.S. Cl. 514/12; 514/563

(57) ABSTRACT

The present invention provides a composition (e.g., a pharmaceutical composition) comprising at least one delivery agent compound and at least one of peptide YY (PYY) and a PYY agonist. Preferably, the composition includes a therapeutically effective amount of peptide YY or the PYY agonist and the delivery agent compound. The composition of the present invention facilitates the delivery of PYY, a PYY agonist, or a mixture thereof and increases its bioavailability compared to administration without the delivery agent compound. PPY and PYY agonists possess activity as agents to reduce nutrient availability, including reduction of food intake

This application claims the benefit of U.S. Provisional Patent Application No. 60/470,905, filed May 14, 2003, U.S. Provisional Patent Application No. 60/471,114, filed May 15, 2003, U.S. Provisional Patent Application No. 60/506,702, filed Sep. 25, 2003, and U.S. Provisional Patent Application No. 60/536,697, filed Jan. 14, 2004, all of which are hereby incorporated by reference.

### FIELD OF THE INVENTION

The present invention relates to compositions for delivering peptide YY (PYY) and PYY agonists to a target. These composition include compounds that are well suited for forming non-covalent mixtures with PYY and PYY agonists for oral administration to animals. Methods for preparation, administration and treatment are also disclosed.

### BACKGROUND OF THE INVENTION

Current antiobesity drugs have limited efficacy and numerous side effects. Crowley, V. E., Yeo, G. S. & O'Rahilly, S., Nat. Rev. Drug Discov 1, 276-86 (2002). With obesity reaching epidemic proportions worldwide, there is a pressing need for the development of adequate therapeutics in this area. In recent years, hormones and neuropeptides involved in the regulation of appetite, body energy expenditure, and fat mass accumulation have emerged as potential antiobesity drugs. McMinn, J. E., Baskin, D. G. & Schwartz, M. W., Obes Rev 1:37-46 (2000), Drazen, D. L. & Woods, S. C., Curr Opin Clin Nutr Metab Care 6:621-629 (2003). At present, however, these peptides require parenteral administration. The prospect of daily injections to control obesity is not very encouraging and may limit the use of these drugs.

One such peptide, PYY, is secreted postprandially by endocrine cells of the distal gastrointestinal tract and acts at the hypothalamus signaling satiety. Batterham, R. L. et al., Nature 418:650-654 (2002). Recent studies have shown that fasting and postprandial PYY levels are low in obese subjects, which may account for their high appetite and food consumption. When administered intravenously, it suppresses appetite and food intake in both lean and obese subjects. Batterham, R. L. et al., N Engl J Med 349:941-948 (2003). Other peptides from the pancreatic peptide (PP) family, like peptide YY fragments (e.g. PYY[3-36]), and PYY agonists (including those not in the PP family) also suppress appetite. Its oral activity, however, is negligible due to its low absorption and rapid degradation in the gastrointestinal tract.

In the delivery to animals of PYY and PYY agonist, barriers are imposed by the body. Examples of physical barriers are the skin, lipid bi-layers and various organ membranes that are relatively impermeable to certain active agents but must be traversed before reaching a target, such as the circulatory system. Chemical barriers include, but are not limited to, pH variations in the gastrointestinal (GI) tract and degrading enzymes.

These barriers are of particular significance in the design of oral delivery systems. Oral delivery of PYY and PYY agonist would be the route of choice for administration to animals if not for such biological, chemical, and physical barriers. These agents may be rapidly rendered ineffective or destroyed in the gastro-intestinal tract by acid hydrolysis, enzymes, and the like. In addition, the size and structure of macromolecular drugs may prohibit absorption. As a result, the oral administration of protein and peptide drugs is challenging due, in part, to their low absorption and rapid degradation.

Earlier methods for orally administering vulnerable pharmacological agents have relied on the co-administration of adjuvants (e.g., resorcinols and non-ionic surfactants such as polyoxyethylene oleyl ether and n-hexadecylpolyethylene ether) to increase artificially the permeability of the intestinal walls, as well as the co-administration of enzymatic inhibitors to inhibit enzymatic degradation. Liposomes have also been described as drug delivery systems for insulin and heparin. However, broad spectrum use of such drug delivery systems is precluded in part because: (1) the systems require toxic amounts of adjuvants or inhibitors; (2) suitable low molecular weight cargos, i.e., active agents, are not available; (3) the systems exhibit poor stability and inadequate shelf life; (4) the systems are difficult to manufacture; (5) the systems fail to protect the active agent (cargo); (6) the systems adversely alter the active agent; or (7) the systems fail to allow or promote absorption of the active agent.

More recently, proteinoid microspheres have been used to deliver pharmaceuticals. See, for example, U.S. Pat. Nos. 5,401,516; 5,443,841; and Re. 35,862. In addition, certain modified amino acids have been used to deliver pharmaceuticals. See, for example, U.S. Pat. Nos. 5,629,020; 5,643,957; 5,766,633; 5,776,888; and 5,866,536.

According to Batterham et al., Nature 418:650-654 (2002), which is hereby incorporated by reference, the peptide YY [3-36] system may provide a therapeutic target for the treatment of obesity.

International Publication No. WO 02/47712 and U.S. patent Publication No. 2002/0141985 disclose methods for treating obesity and diabetes with peptide YY and peptide YY agonists, such as peptide YY[3-36].

However, there is still a need for simple, inexpensive delivery systems for deliverying peptide YY and PYY agonists.

There is a need for a non-invasive route of delivering the peptide YY[3-36], preferably orally, to ensure patient compliance.

### SUMMARY OF THE INVENTION

The present invention provides a composition (e.g., a pharmaceutical composition) comprising (a) at least one delivery agent compound and (b) peptide YY (PYY), a PYY agonist, or a mixture thereof. Preferably, the composition includes a therapeutically effective amount of peptide YY and/or the PYY agonist and the delivery agent compound. The composition of the present invention facilitates the delivery of PYY and/or the PYY agonist and increases its bioavailability compared to administration without the delivery agent compound. PPY and PYY agonists possess activity as agents to reduce nutrient availability, including reduction of food intake.

Preferred delivery agent compounds include, but are not limited to, N-(8-[2-hydroxybenzoyl]amino)caprylic acid and N-(10-[2-hydroxybenzoyl]amino)decanoic acid and salts thereof, and solvates and hydrates thereof. In a preferred embodiment, the salt is the sodium salt, such as the monosodium salt.

In one preferred embodiment, the composition comprises peptide YY, a PYY agonist, or a mixture thereof, and at least one delivery agent of the following structure or a salt thereof: 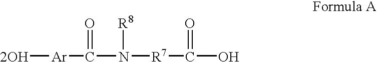
wherein
Ar is phenyl or naphthyl;
Ar is optionally substituted with one or more of —OH, halogen, C₁-C₄ alkyl, C₁-C₄ alkenyl, C₁-C₄ alkoxy or C₁-C₄ haloalkoxy;
R⁷ is selected from C₄-C₂₀ alkyl, C₄-C₂₀ alkenyl, phenyl, naphthyl, (C₁-C₁₀ alkyl) phenyl, (C₁-C₁₀ alkenyl)phenyl, (C₁-C₁₀ alkyl) naphthyl, (C_{1-C}₁₀ alkenyl) naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);
R⁸ is selected from hydrogen, C₁ to C₄ alkyl, C₂ to C₄ alkenyl, C₁ to C₄ alkoxy, and C₁-C₄ haloalkoxy;
R⁷ is optionally substituted with C₁ to C₄ alkyl, C₂ to C₄ alkenyl, C₁ to C₄ alkoxy, C₁-C₄ haloalkoxy, —OH, —SH, —CO₂R⁹, or any combination thereof;
R⁹ is hydrogen, C₁ to C₄ alkyl, or C₂ to C₄ alkenyl.
R⁷ is optionally interrupted by oxygen, nitrogen, sulfur or any combination thereof; with the proviso that the compounds are not substituted with an amino group in the position alpha to the acid group.

In another preferred embodiment, the composition comprises peptide YY, a PYY agonist, or a mixture thereof, and at least one delivery agent of the following structure or a salt thereof: 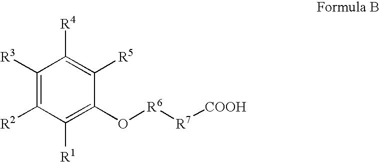
wherein
R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹ (R¹²)⁻;
R⁵ is H, —OH, —NO₂, halogen, —CF₃, —NR¹⁴R¹⁵, —N⁺R¹⁴R¹⁵R¹⁶ (R¹³)⁻, amide, C₁-C₁₂ alkoxy, C₁-C₁₂ alkyl, C₂-C₁₂ alkenyl, carbamate, carbonate, urea, or —C(O)R¹⁸;
R⁵ is optionally substituted with halogen, —OH, —SH, or —COOH;
R⁵ is optionally interrupted by O, N, S, or —C(O)—;
R⁶ is a C₁-C₁₂ alkylene, C₂-C₁₂ alkenylene, or arylene;
R⁶ is optionally substituted with a C₁-C₄ alkyl, C₂-C₄ alkenyl, C₁-C₄ alkoxy, —OH, —SH, halogen, —NH₂, or —CO₂R⁸;
R⁶ is optionally interrupted by O or N;
R⁷ is a bond or arylene;
R⁷ is optionally substituted with —OH, halogen, —C(O)CH₃, —NR¹⁰R¹¹, or —N⁺R¹⁰R¹¹R¹² (R¹³)⁻;
each occurrence of R⁸ is independently H, C₁-C₄ alkyl, C₂-C₄ alkenyl, or —NH₂;
R⁹, R¹⁰, R¹¹, and R¹² independently H or C₁-C₁₀ alkyl;
R¹³ is a halide, hydroxide, sulfate, tetrafluoroborate, or phosphate;
R¹⁴, R¹⁵ and R¹⁶ are independently H, C₁-C₁₀ alkyl, C₁-C₁₀ alkyl substituted with —COOH, C₂-C₁₂ alkenyl, C₂-C₁₂ alkenyl substituted with —COOH, or —C(O)R¹⁷;
R¹⁷ is —OH, C₁-C₁₀ alkyl, or C₂-C₁₂ alkenyl; and
R¹⁸ is H, C₁-C₆ alkyl, —OH, —NR¹⁴R¹⁵, or N⁺R¹⁴R¹⁵R¹⁶(R¹³).

Optionally, when R¹, R², R³, R⁴, and R⁵ are H, and R⁷ is a bond then R⁶ is not a C₁-C₆, C₉ or C₁₀ alkyl.

Optionally,when R¹, R², R³, and R⁴ are H, R⁵ is —OH, R⁷ is a bond then R⁶ is not a C₁-C₃ alkyl.

Optionally,when at least one of R¹, R², R³, and R⁴ is not H, R⁵ is —OH, R⁷ is a bond, then R⁶ is not a C₁-C₄ alkyl.

Optionally,when R¹, R², and R³ are H, R⁴ is —OCH₃, R⁵ is —C(O)CH₃, and R⁶ is a bond then R⁷ is not a C₃ alkyl.

Optionally,when R¹, R², R⁴, and R⁵ are H, R³ is —OH, and R⁷ is a bond then R⁶ is not a methyl.

In yet another embodiment the composition comprises peptide YY, PYY agonist, or a mixture thereof and at least one delivery agent of the following structure or a salt thereof: 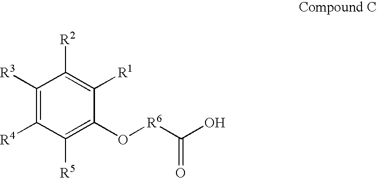
wherein
R¹, R², R³, R⁴ and R⁵ are independently H, —CN, —OH, —OCH₃, or halogen, at least one of R¹, R², R³, R⁴ and R⁵ being —CN; and
R⁶ is a C₁-C₁₂ linear or branched alkylene, alkenylene, arylene, alkyl(arylene) or aryl(alkylene).

According to one embodiment, when R¹ is —CN, R⁴is H or —CN, and R², R³, and R⁵ are H, then R⁶ is not methylene ((CH₂)₁).

Also provided is a dosage unit form (e.g., an oral dosage unit form) comprising the composition of the present invention. The dosage unit form may be in the form of a liquid or a solid, such as a tablet, capsule or particle, including a powder or sachet.

Another embodiment is a method for administering peptide YY, a PYY agonist, or a mixture thereof to an animal in need thereof, by administering the composition or dosage unit form(s) of the present invention to the animal. The preferred route of administration is oral.

Another embodiment is a method for administering peptide YY, a PYY agonist, or a mixture thereof to an animal in need thereof, by administering the composition or dosage unit form(s) of the present invention to the animal in a manner to minimize or prevent formation of antibodies to the peptide YY and/or a PYY agonist.

Yet another embodiment is a method of losing weight in an animal (such as a human) in need thereof by administering an effective amount of the composition or dosage unit form(s) of the present invention to the animal. In other words, an effective amount of the delivery agent compound to facilitate the delivery of the PYY or PYY agonist and an effective amount (e.g., a therapeutically effective amount) of PYY or PYY agonist are administered.

Yet another embodiment is a method of treating obesity in an animal (such as a human) in need thereof by administering an effective amount of the composition of the present invention to the animal.

Yet another embodiment is a method for treating conditions or disorders which can be alleviated by reducing nutrient availability in an animal (such as a human) by administering to the animal a therapeutically effective amount of the composition or dosage unit form(s) of the present invention. Such conditions and disorders, include but are not limited to, hypertension, dyslipidemia, cardiovascular risk, an eating disorder, insulin-resistance, obesity, and diabetes mellitus.

Yet another embodiment is a method of improving the lipid profile in an animal (such as a human) by administering to the animal an effective amount of the composition or dosage unit form(s) of the present invention.

Yet another embodiment is a method of preparing a composition of the present invention by mixing at least one delivery agent compound and at least one of peptide YY and a PYY agonist.

### BRIEF DESCRIPTION OF THE DRAWINGS

FIG. 1 is a graph of the serum concentrations (pg/ml±standard error) of PYY[3-36] after oral administration of PYY[3-36] with and without the delivery agent SNAC by the procedure described in Example 1 versus time.

FIG. 2 is a graph of the serum concentrations (pg/ml±standard error) of PYY[3-36] after oral administration of PYY[3-36] with and without the delivery agent SNAD by the procedure described in Example 1 versus time.

FIG. 3 is a graph of the serum concentrations (pg/ml±standard error) of PYY[3-36] after intraperitoneal administration of PYY[3-36] without a delivery agent compound by the procedure described in Example 2 versus time.

FIG. 4 is a graph of the serum concentrations (pg/ml±standard error) of PYY[3-36] after oral administration of PYY[3-36] to rats with various delivery agents, delivery agent 3 alone and PYY with Mannitol by the procedure in Example 3 versus time.

FIG. 5 is a graph of the serum concentrations (pg/ml±standard error) of PYY[3-36] after oral administration of PYY[3-36] to non-human primates with delivery agent 1 by the procedure in Example 4 versus time.

FIG. 6 is a graph of the serum concentrations of PYY[3-36] over time after oral administration of PYY[3-36] in combination with SNAD by the procedure described in Example 3c.

FIG. 7 is a graph of food intake during a 4-day treatment of Male Sprague Dawley rats with PYY[3-36] in combination with SNAD, as compared to a placebo by the procedure as described in Example 5.

FIG. 8a is a graph of the cumulative weight gain during a 7-day treatment of Male Sprague Dawley rats with PYY[3-36] in combination with SNAD, as compared to a placebo by the procedure described in Example 6.

FIG. 8b is a graph of the cumulative food intake and weight gain during a 7-day treatment of Male Sprague Dawley Rats with PYY[3-36] in combination with SNAD, as compared to a placebo by the procedure described in Example 6.

### DETAILED DESCRIPTION OF THE INVENTION

Definitions

The term “hydrate” as used herein includes, but is not limited to, (i) a substance containing water combined in the molecular form and (ii) a crystalline substance containing one or more molecules of water of crystallization or a crystalline material containing free water.

The term “solvate” as used herein includes, but is not limited to, a molecular or ionic complex of molecules or ions of a solvent with molecules or ions of the delivery agent compound or salt thereof, or hydrate or solvate thereof.

The term “delivery agent” refers to any of the delivery agent compounds disclosed herein.

The term “SNAC” refers to the monosodium salt of N-(8-[2-hydroxybenzoyl]-amino)caprylic acid.

The term “SNAD” refers to the monosodium salt of N-(10-[2-hydroxybenzoyl]-amino)decanoic acid. The term “disodium salt of SNAD” refers to the disodium salt of N-(10-[2-hydroxybenzoyl]-amino)decanoic acid.

An “effective amount of PYY, PYY agonist, or a mixture thereof” is an amount of the PYY, the PYY agonist, or mixture thereof which is effective to treat or prevent a condition in a living organism to whom it is administered over some period of time, e.g., provides a therapeutic effect during a desired dosing interval.

An “effective amount of delivery agent” is an amount of the delivery agent which enables and/or facilitates the absorption of a desired amount of PYY or PYY agonist via any route of administration (such as those discussed in this application including, but not limited to, the oral (e.g., across a biological membrane in the gastrointestinal tract), nasal, pulmonary, dermal, buccal, vaginal, and/or ocular route).

The term “AUC” as used herein, means area under the plasma concentration-time curve, as calculated by the trapezoidal rule over the complete dosing interval, e.g., 24-hour interval.

The term “mean”, when preceding a pharmacokinetic value (e.g., mean Peak) represents the arithmetic mean value of the pharmacokinetic value unless otherwise specified.

As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different-reagents, reference to “an antibody” includes one or more of such different antibodies, and reference to “the method” includes reference to equivalent steps and methods known to those of ordinary skill in the art that could be modified or substituted for the methods described herein.

The term “about” generally means within 10%, preferably within 5%, and more preferably within 1% of a given value or range.

The terms “alkyl” and “alkenyl” as used herein include linear and branched alkyl and alkenyl substituents, respectively.

The term “patient” as used herein refers to a mammal and preferably a human.

The phrase “pharmaceutically acceptable” refers to additives or compositions that are physiologically tolerable and do not typically produce an allergic or similar untoward reaction, such as gastric upset, dizziness and the like, when administered to a mammal. PYY and PYY Agonists

By “peptide YY” or “PYY” is meant a Peptide YY polypeptide obtained or derived from any species. Thus, the term “PYY” includes both the human full length, 36 amino acid peptide as set forth in SEQ ID NO: 2 of International Publication No. WO 02/47712 (which is the PCT counterpart to U.S. patent Publication No. 2002/0141985, which is hereby incorporated by reference) and Tatemoto, Proc Natl Acad Sci U.S.A. 79:2514-8, 1982, and species variations of PYY, including e.g., murine, hamster, chicken, bovine, rat, and dog PYY, for example. By “PYY agonist” is meant any compound which elicits an effect of PYY to reduce nutrient availability, for example a compound (1) having activity in the food intake, gastric emptying, pancreatic secretion, or weight loss assays described in Examples 1, 2, 5, or 6 of WO 02/47712 and U.S. patent Publication No. 2002/0141985, and (2) which binds specifically in a Y receptor assay (Example 10 of WO 02/47712 and U.S. patent Publication No. 2002/0141985) or in a competitive binding assay with labeled PYY or PYY [3-36] from certain tissues having an abundance of Y receptors, including e.g., area postrema (Example 9 of WO 02/47712 and U.S. patent Publication No. 2002/0141985), wherein the PYY agonist is not pancreatic polypeptide. Preferably, PYY agonists would bind in such assays with an affinity of greater than about 1 μM, and more preferably with an affinity of greater than about 1 to about 5 nM.

Such agonists can comprise a polypeptide having a functional PYY domain, an active fragment of PYY, or a chemical or small molecule. PYY agonists may be peptide or nonpeptide compounds, and include “PYY agonist analogs,” which refer to any compound structurally similar to a PYY that have PYY activity typically by virtue of binding to or otherwise directly or indirectly interacting with a PYY receptor or other receptor or receptors with which PYY itself may interact to elicit a biological response. Such compounds include derivatives of PYY, fragments of PYY, extended PYY molecules having more than 36 amino acids, truncated PYY molecules having less than 36 amino acids, and substituted PYY molecules having one or more different amino acids, or any combination of the above. Such compounds may also be modified by processes such as pegylation, amidation, glycosylation, acylation, sulfation, phosphorylation, acetylation and cyclization.

One such PYY agonist analog is PYY [3-36], identified as SEQ ID NO: 3 of WO 02/47712 and U.S. patent Publication No. 2002/0141985; Eberlein, Eysselein et al., Peptides 10:797-803 (1989); and Grandy, Schimiczek et al., Regul Pept 51:151-9 (1994). Polypeptides with numbers in brackets refer to truncated polypeptides having the sequence of the full length peptide over the amino acid positions in the brackets. Thus, PYY [3-36] has a sequence identical to PYY over amino acids 3 to 36. PYY[3-36] contains approximately 40% of total peptide YY-like immunoreactivity in human and canine intestinal extracts and about 36% of total plasma peptide YY immunoreactivity in a fasting state to slightly over 50% following a meal. It is apparently a dipeptidyl peptidase-IV (DPP4) cleavage product of peptide YY. Peptide YY[3-36] is reportedly a selective ligand at the Y2 and Y5 receptors, which appear pharmacologically unique in preferring N-terminally truncated (i.e. C terminal fragments of) neuropeptide Y analogs. A PYY agonist may bind to a PYY receptor with higher or lower affinity, demonstrate a longer or shorter half-life in vivo or in vitro, or be more or less effective than native PYY.

Other suitable PYY agonists include those described in International Publication No. WO 98/20885, which is hereby incorporated by reference.

By “condition or disorder which can be alleviated by reducing caloric (or nutrient) availability” is meant any condition or disorder in an animal that is either caused by, complicated by, or aggravated by a relatively high nutrient availability, or that can be alleviated by reducing nutrient availability, for example by decreasing food intake. Such conditions or disorders include, but are not limited to, obesity, diabetes, including type 2 diabetes, eating disorders, and insulin-resistance syndromes.

In one aspect, the invention provides a method of treating obesity in an obese or overweight animal by administering a therapeutically effective amount of PYY, a PYY agonist, or a mixture thereof with at least one delivery agent compound. While “obesity” is generally defined as a body mass index over 30, for purposes of this disclosure, any subject, including those with a body mass index of less than 30, who needs or wishes to reduce body weight is included in the scope of “obese.” Subjects who are insulin resistant, glucose intolerant, or have any form of diabetes mellitus (e. g., type 1, 2 or gestational diabetes) can benefit from this method.

In other aspects, the invention features methods of reducing food intake, treating diabetes mellitus, and improving lipid profile (including reducing LDL cholesterol and triglyceride levels and/or changing HDL cholesterol levels) comprising administering to a subject a therapeutically effective amount of PYY, a PYY agonist, or a mixture thereof with at least one delivery agent compound. In a preferred embodiment, the methods of the invention are used to treat conditions or disorders which can be alleviated by reducing nutrient availability in a subject in need thereof, comprising administering to said subject a therapeutically effective amount of PYY, a PYY agonist, or a mixture thereof with at least one delivery agent compound. Such conditions and disorders include, but are not limited to, hypertension, dyslipidemia, cardiovascular disease, eating disorders, insulin-resistance, obesity, and diabetes mellitus of any kind.

In the methods of the invention, preferred PYY agonists are those having a potency in one of the assays described in WO 02/47712 and U.S. patent Publication No. 2002/0141985 (preferably food intake, gastric emptying, pancreatic secretion, or weight reduction assays) which is greater than the potency of NPY in that same assay.

In one embodiment, for treatment of all conditions and disorders described herein, a preferred PYY agonist is PYY [3-36], and is administered (e.g. peripherally) at a dose of about 1 pg to about 5 mg per day in single or divided doses. Alternatively PYY[3-36] may be administered on the basis of the recipients total body weight in an amount of about 0.01 μg/kg to about 500 μg/kg, or about 0.05 μg/kg to about 250 μg/kg, or less than about 50 μg/kg, per day in a single or divided doses. Dosages in these ranges will vary with the potency of each agonist, of course, and are readily determined by one of skill in the art.

In the methods of the present invention, PYY's and PYY agonists with the delivery agent compound may be administered separately or together with one or more other compounds and compositions that exhibit a long term or short-term action to reduce nutrient availability, including, but not limited to other compounds and compositions that comprise an amylin or amylin agonist, a cholecystokinin (CCK) or CCK agonist, a leptin (OB protein) or leptin agonist, an exendin or exendin agonist, or a GLP-1 or GLP-1 agonist. Suitable amylin agonists include, for example, [25,28,29Pro-]-human amylin (also known as “pramlintide”, and described in U.S. Pat. Nos. 5,686,511 and 5,998,367), calcitonin (e.g., salmon calcitonin), including those described in U.S. Pat. No. 5,739,106, which is hereby incorporated by reference. The CCK used is preferably CCK octopeptide (CCK-8). Leptin is discussed in, for example, Pelleymounter, C. et al., Science 269: 540-543 (1995), Halaas, G. et al., Science 269: 543-6 (1995) and Campfield, S. et al., Science 269: 546-549 (1995). Suitable CCK agonist includes those described in U.S. Pat. No. 5,739,106, which is hereby incorporated by reference. Suitable exendins include exendin-3 and exendin-4, and exendin agonist compounds include, for example, those described in PCT Publications WO 99/07404, WO 99/25727, and WO 99/25728, all of which are hereby incorporated by reference. According to one embodiment, the composition of the present invention includes at least one delivery agent compound, PYY, a PYY agonist, or a mixture thereof, at least one amylin agonist, and a CCK agonist. Suitable combinations of amylin agonist and CCK agonist include, but are not limited to, those described in U.S. Pat. No. 5,739,106, which is hereby incorporated by reference.

PYY and PYY[3-36] are C-terminally amidated when expressed physiologically, but need not be for the purposes of the present invention. These peptides may also have other posttranslational modifications.

PYY and peptide-based PYY agonists described herein may be prepared using standard recombinant expression or chemical peptide synthesis techniques known in the art, e. g., using an automated or semiautomated peptide synthesizer. PYY as described herein include any morphologies of PYY [3-36], including those obtained by lyophilization, crystallization, reconstitution, spray drying, and super critical fluid processing.

Solid phase peptide synthesis may be carried out with an automatic peptide synthesizer (e. g., Model 430A, Applied Biosystems Inc., Foster City, Calif.) using the NMP/HOBt (Option 1) system and tBoc or Fmoc chemistry (see, Applied Biosystems User's Manual for the ABI 430A Peptide Synthesizer, Version 1.3B Jul. 1, 1988, 6: 4970, Applied Biosystems, Inc., Foster City, Calif.) with capping. Peptides may be also be assembled using an Advanced Chem Tech Synthesizer (Model MPS 350, Louisville, Ky.). Peptides may be purified by RP-HPLC (preparative and analytical) using, e.g., a Waters Delta Prep 3000 system and a C4, C8 or C18 preparative column (10p, 2.2×25 cm; Vydac, Hesperia, Calif.).

Peptide compounds useful in the invention may also be prepared using recombinant DNA techniques, using methods now known in the art. See, e. g., Sambrook et al., Molecular Cloning: A Laboratory Manual, 2d Ed., Cold Spring Harbor (1989). Non-peptide compounds useful in the present invention may be prepared by art-known methods. For example, phosphate-containing amino acids and peptides containing such amino acids, may be prepared using methods known in the art. See, e. g., Bartlett and Landen, Biorg Chem. 14: 356-377 (1986).

The compositions useful in the invention can be provided as parenteral compositions for e. g., injection or infusion. For example, they may be suspended in an aqueous carrier, for example, in an isotonic buffer solution at a pH of about 3.0 to about 8.0. Useful buffers include sodium citrate-citric acid and sodium phosphate-phosphoric acid, and sodium acetate/acetic acid buffers. A form of repository or “depot” slow release preparation may be used so that therapeutically effective amounts of the preparation are delivered into the bloodstream over many hours or days following transdermal injection or delivery.

Since PYY and many PYY agonists are amphoteric, they may be utilized as free bases, as acid addition salts or as metal salts. The salts preferably are pharmaceutically acceptable, and these will include metal salts, particularly alkali and alkaline earth metal salts, e, g., potassium or sodium salts. A wide variety of pharmaceutically acceptable acid addition salts are available. Such products are readily prepared by procedures well known to those skilled in the art.

Therapeutically effective amounts of a PYY or a PYY agonist for use in reducing nutrient availability are those that suppress appetite at a desired level. As will be recognized by those in the field, an effective amount of therapeutic agent will vary with many factors including the age and weight of the patient, the patient's physical condition, the blood sugar level, the weight level to be obtained, and other factors.

The effective daily appetite-suppressing dose of PYY, a PYY agonist, or a mixture thereof may be in the range of about 1 to 30 ptg to about 50 mg/day, or about 10 to 30 μg to about 20 mg/day and or about 5 to 100 μg to about 10 mg/day, or about 5 μg to about 5 mg/day, for a 50 kg patient. Effective amounts of PYY or a PYY agonist may be administered in a single or divided doses. The dosages may be between about 0.01 to about 1 mg/kg/dose. The exact dose to be administered may be determined by one of skill in the art and is dependent upon the potency of PYY, PYY agonist, or mixture thereof, as well as upon the age, weight and condition of the individual. Administration should begin whenever the suppression of nutrient availability, food intake, weight, blood glucose or plasma lipid lowering is desired, for example, at the first sign of symptoms or shortly after diagnosis of obesity, diabetes mellitus, or insulin resistance syndrome.

Screening for Additional PYY Agonists

Other PYY agonists can be identified by using the receptor binding assays described below (e. g., in Examples 9 and 10 of WO 02/47712 and U.S. patent Publication No. 2002/0141985) or known in the art in combination with the physiological screens described in the examples in WO 02/47712 and U.S. patent Publication No. 2002/0141985. Potential PYY agonists can be compared with the activity of PYY or PYY [3-36].

Alternatively, once one or more PYY-preferring (Y7) receptors have been characterized and cloned, alternative assays and high throughput screens can be implemented as discussed below or known in the art. Y7 receptors are those with an affinity for PYY or PYY [3-36] greater than their affinity for neuropeptide Y (NPY). Methods of screening for compounds which modulate PYY receptor activity comprise contacting test compounds with PYY receptors and assaying for the presence of a complex between the compound and the PYY receptors. In such assays, the test ligand is typically labeled. After suitable incubation, free ligand is separated from that present in bound form, and the amount of free or uncomplexed label is a measure of the ability of the particular compound to bind to the PYY receptors.

Alternatively, bound labeled ligand may be measured (e. g., using expressed membrane bound Y7 receptors).

High throughput screening for PYY agonists having suitable binding affinity to PYY receptors may be employed. For example, large numbers of different small peptide test compounds are synthesized on a solid substrate. The peptide test compounds are contacted with the PYY receptor and washed. Bound PYY receptor is then detected by methods well known in the art. Purified test compounds can also be coated directly onto plates for use in the aforementioned drug screening techniques. In addition, if the test compounds are proteins, antibodies can be used to capture the protein and immobilize it on the solid support by any means known in the art.

Competitive screening assays may be used in which neutralizing antibodies capable of binding a polypeptide of the invention specifically compete with a test compound for binding to the polypeptide. In this manner, the antibodies can be used to detect the presence of any peptide that shares one or more antigenic determinants with a PYY agonist. Radiolabeled competitive binding studies are described in Lin, A. H. et al., Antimicrobial Agents and Chemotherapy 41(10): 2127-2131 (1997), the disclosure of which is incorporated herein by reference in its entirety.

Delivery Agent Compounds

The delivery agent compound may be any of those described in U.S. Pat. Nos. 5,650,386 and 5,866,536 and International Publication Nos. WO94/23767, WO95/11690, WO95/28920, WO95/28838, WO96/10396, WO96/09813, WO96/12473, WO96/12475, WO96/30036, WO96/33699, WO97/31938, WO97/36480, WO98/21951, WO98/25589, WO98/34632, WO98/49135, WO99/16427, WO00/06534, WO00/07979, WO00/40203, WO00/46182, WO00/47188, WO00/48589, WO00/50386, WO00/59863, WO00/59480, WO01/32130, WO01/32596, WO01/34114, WO01/44199, WO01/51454, WO01/70219, WO01/92206, WO02/02509, WO02/15959, WO02/16309, WO02/20466, WO02/19969, WO02/070438, WO03/026582, WO02/100338, WO03/045306, and WO0326582, all of which are hereby incorporated by reference.

Non-limiting examples of delivery agent compounds include N-(8-[2-hydroxybenzoyl]-amino)caprylic acid, N-(10-[2-hydroxybenzoyl]-amino)decanoic acid, 8-(2-hydroxy-4-methoxybenzoylamino)octanoic acid, 8-(2,6-dihydroxybenzoylamino)octanoic acid, 8-(2-hydroxy-5-bromobenzoylamino)octanoic acid, 8-(2-hydroxy-5-chlorobenzoylamino)octanoic acid, 8-(2-hydroxy-5-iodobenzoylamino)octanoic acid, 8-(2-hydroxy-5-methylbenzoylamino)octanoic acid, 8-(2-hydroxy-5-fluorobenzoylamino)octanoic acid, 8-(2-hydroxy-5-methoxybenzoylamino)octanoic acid, 8-(3-hydroxyphenoxy)octanoic acid, 8-(4-hydroxyphenoxy)octanoic acid, 6-(2-cyanophenoxy)hexanoic acid, 8-(2-Hydroxyphenoxy)octyl-diethanolamine, 8-(4-hydroxyphenoxy)octanoate, 8-(4-hydroxyphenoxy)octanoate, 8-(2-hydroxy-4-methoxybenzoylamino)octanoic acid, 8-(2-hydroxy-5-methoxybenzoylamino)-octanoic acid., and salts thereof. Preferred salts include, but are not limited to, monosodium and disodium salts.

The delivery agent compounds may be in the form of the carboxylic acid or pharmaceutically acceptable salts thereof, such as sodium salts, and hydrates and solvates thereof. The salts may be mono- or multi-valent salts, such as monosodium salts and disodium salts. The delivery agent compounds may contain different counter ions chosen for example due to their effect on modifying the dissolution profile of the carrier.

The delivery agent compounds may be prepared by methods known in the art, such as those discussed in the aforementioned publications (e.g., International Publication Nos. WO 98/34632, WO 00/07979, WO 01/44199, WO 01/32596, WO 02/20466, and WO 03/045306). SNAC, SNAD, and the free acid and other salts thereof may be prepared by any method known in the art, such as those described in U.S. Pat. Nos. 5,650,386 and 5,866,536.

Salts of the delivery agent compounds of the present invention may be prepared by methods known in the art. For example, sodium salts may be prepared by dissolving the delivery agent compound in ethanol and adding aqueous sodium hydroxide.

The delivery agent compound may be purified by recrystallization or by fractionation on one or more solid chromatographic supports, alone or linked in tandem. Suitable recrystallization solvent systems include, but are not limited to, acetonitrile, methanol, and tetrahydrofuran. Fractionation may be performed on a suitable chromatographic support such as alumina, using methanol/n-propanol mixtures as the mobile phase; reverse phase chromatography using trifluoroacetic acid/acetonitrile mixtures as the mobile phase; and ion exchange chromatography using water or an appropriate buffer as the mobile phase. When anion exchange chromatography is performed, preferably a 0-500 mM sodium chloride gradient is employed.

Delivery Systems

The composition of the present invention comprises one or more delivery agent compounds of the present invention and/or one or more of PYY and PYY agonists. The delivery agent compound and PYY and/or the PYY agonists are typically mixed prior to administration to form an administration composition.

The composition may include one or more food-intake-reducing, plasma glucose-lowering or plasma lipid-altering agents, such as an amylin, an amylin agonist, a CCK, or CCK agonist, or a leptin or leptin agonist, or an exendin or exendin agonist.

The administration compositions may be in the form of a liquid. The solution medium may be water, 25% aqueous propylene glycol, or phosphate buffer. Other dosing vehicles include polyethylene glycol. Dosing solutions may be prepared by mixing a solution of the delivery agent compound with a solution of the active agent, just prior to administration. Alternately, a solution of the delivery agent compound (or PYY, PYY agonist, or mixture thereof) may be mixed with the solid form of PYY or the PYY agonist (or delivery agent compound). The delivery agent compound and PYY, PYY agonist, or mixture thereof may also be mixed as dry powders. The delivery agent compound and PYY, PYY agonist, or mixture thereof can also be admixed during the manufacturing process.

The dosing solutions may optionally contain additives such as phosphate buffer salts, citric acid, glycols, or other dispersing agents. Stabilizing additives may be incorporated into the solution, preferably at a concentration ranging between about 0.1 and 20% (w/v).

The administration compositions may alternately be in the form of a solid, such as a tablet, capsule or particle, such as a powder or sachet. Solid dosage forms may be prepared by mixing the solid form of the compound with the solid form of PYY, PYY agonist, or mixture thereof. Alternately, a solid may be obtained from a solution of compound and PYY, PYY agonist, or mixture thereof by methods known in the art, such as freeze-drying (lyophilization), precipitation, crystallization and solid dispersion. Alternatively, the administration can be a semi-solid, in the form of a gel, paste, colloid, gelatin, emulsion, suspension and the like.

The administration compositions of the present invention may also include one or more enzyme inhibitors. Such enzyme inhibitors include, but are not limited to, compounds such as actinonin or epiactinonin and derivatives thereof. Other enzyme inhibitors include, but are not limited to, aprotinin (Trasylol) and Bowman-Birk inhibitor.

The amount of PYY and/or the PYY agonist used in an administration composition of the present invention is an amount effective to treat the target indication. However, the amount can be less than that amount when the composition is used in a dosage unit form because the dosage unit form may contain a plurality of delivery agent compound/PYY or the PYY agonist compositions or may contain a divided effective amount. The total effective amount can then be administered in cumulative units containing, in total, an effective amount of PYY, PYY agonist, or mixture thereof. Moreover, those skilled in the filed will recognize that an effective amount of PYY, PYY agonist, or mixture thereof will vary with many factors including the age and weight of the patient, the patient's physical condition, the blood sugar level, the weight level to be obtained, as well as other factors.

The total amount to be used of PYY or the PYY agonist can be determined by methods known to those skilled in the art. However, because the compositions of the invention may deliver PYY or the PYY agonist more efficiently than compositions containing PYY or the PYY agonist alone, lower amounts of PYY or the PYY agonist than those used in prior dosage unit forms or delivery systems can be administered to the subject, while still achieving the same blood levels and/or therapeutic effects.

According to one embodiment the amount of PYY, PYY agonist, or mixture thereof administered with the delivery agent is an amount sufficient to suppress appetite to a desired level. The effective daily appetite-suppressing dose of PYY, a PYY agonist, or a mixture thereof generally ranges from about 1 μg about 5 mg per day in single or divided doses, preferably from about 5 μg to about 2 mg/day, and more preferably from about 5 μg to 500 μg/day for a 50 kg patient. Preferably the dosage forms of the present invention consist from about 0.01 and about 10 μg/kg/dose of PYY, a PYY agonist, or a mixture thereof.

The present invention also includes pharmaceutical compositions and dosage forms which include the aforementioned amounts of PYY, a PYY agonist, or a mixture thereof and at least one delivery agent

Generally an effective amount of delivery agent to facilitate the delivery of PYY and/or the PYY agonist is administered with PYY, PYY agonist, or a mixture thereof. Generally the amount of delivery agent to PYY, PYY agonist, or mixture thereof, on a molar basis ranges from about 25000:1 to about 50:1, preferably from about 8000:1 to about 100:1 and most preferably from about 4000:1 to about 300:1.

The presently disclosed delivery agent compounds facilitate the delivery of PYY, a PYY agonist, or a mixture thereof, particularly in oral, intranasal, sublingual, intraduodenal, subcutaneous, buccal, intracolonic, rectal, vaginal, mucosal, pulmonary, transdermal, intradermal, parenteral, intraperitoneal, intravenous, intramuscular and ocular systems, as well as traversing the blood-brain barrier. The compositions and dosage unit forms of the present invention can be administered by any of the aforementioned routes.

The compositions and dosage unit form of the present invention when administered orally to a human can achieve known therapeutic levels of PYY[3-36] in the body, such as those enumerated in Batterham et al., Nature 418:650-654(2002).

Dosage unit forms can also include any one or combination of excipients, diluents, disintegrants, lubricants, plasticizers, colorants, flavorants, taste-masking agents, sugars, sweeteners, salts, and dosing vehicles, including, but not limited to, water, 1,2-propane diol, ethanol, olive oil, or any combination thereof.

The compounds and compositions of the subject invention are useful for administering biologically or chemically active agents to any animals, including but not limited to birds such as chickens; fish, reptiles, mammals, such as rodents, cows, pigs, dogs, cats, primates, and particularly humans, and insects.

### EXAMPLES

The following examples illustrate the invention without limitation. All parts are given by weight unless otherwise indicated.

### Example 1

### Liquid Oral Delivery of PYY[3-36] in Rats

Oral gavage (PO) dosing solutions of delivery agent compound and Peptide YY residues 3-36 (PYY[3-36]) (available from Bachem California Inc. of Torrance, Calif.) in deionized water were prepared as follows.

The dosing solution of Delivery Agent 1 (SNAC) and PYY[3-36] was prepared as follows. SNAC monosodium salt, solid was dissolved in water. The pH of this solution was close to pH 7.5, so no pH adjustments were done. Aliquots of this SNAC solution were mixed with aliquots of a PYY solution, which was at pH 7.5. Solutions of 100 or 200 mg/kg SNAC and 0.1 or 0.5 mg/kg PYY[3-36] were prepared by this procedure. The final pH of these solutions was 7.5.

The dosing solution of the monosodium salt of Delivery Agent 2 (SNAD) and PYY[3-36] was prepared as follows. SNAD disodium salt in solid form was dissolved in water. The pH of the resulting solution was 11.1. The pH was then lowered to 7.7 by adding HCl (5N). Then aliquots of the SNAD solution were mixed with aliquots of a PYY[3-36] solution, which was at pH 7.5. Solutions of 100 or 200 mg/kg SNAD and 0.1 or 0.5 mg/kg PYY[3-36] were prepared by this procedure. The final pH of these solutions varied between 7.5 and 7.6.

The dosing solution of the monosodium salts of Delivery Agents 4 through 15 and PYY[3-36] were prepared as follows. Each delivery agent compound (as the free acid or monosodium salts) was dissolved in water. The pH was adjusted to approximately 7.5 by adding HCl (5N) and NaOH (5N) as needed. Then aliquots of the Delivery Agent solution were mixed with aliquots of a PYY[3-36] solution, which was at pH 7.5. Solutions of 200 mg/kg of Delivery Agent and 0.3 mg/kg PYY[3-36] were prepared by this procedure. The final pH of these solutions was approximately 7.5.

The typical dosing and sampling protocols were as follows. Male Sprague-Dawley rats weighing between 240-320 g were fasted up to a maximum 24 hours before the experiments and administered ketamine (44 mg/kg) and thorazine (1.5 mg/kg) by intramuscular injection before the test article administration. Afterwards, the anesthetized animals were administered the test article by oral gavage. A dosing group of five animals was administered one of the dosing solutions. For oral gavage (PO), an 11 cm Rusch 8 French catheter was adapted to a 1 ml syringe with a pipette tip. The syringe was filled with dosing solution by drawing the solution through the catheter, which was then wiped dry. The catheter was placed down the esophagus leaving 1 cm of tubing past the incisors. The dosing solution was administered by pressing the syringe plunger.

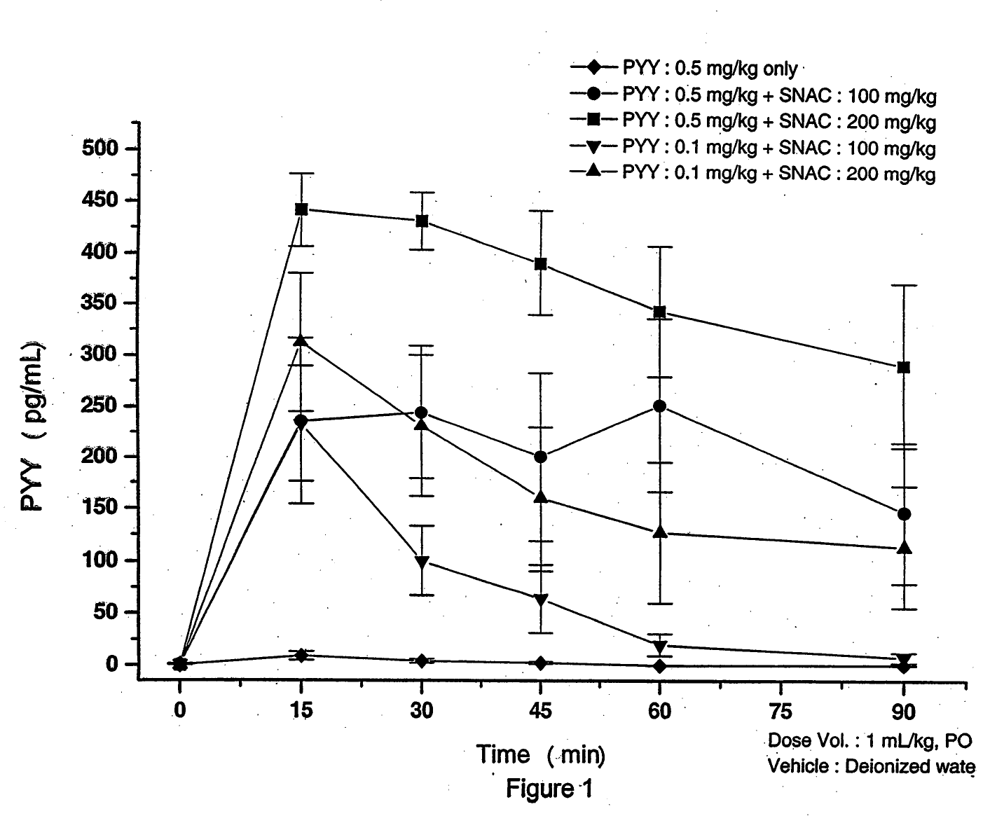

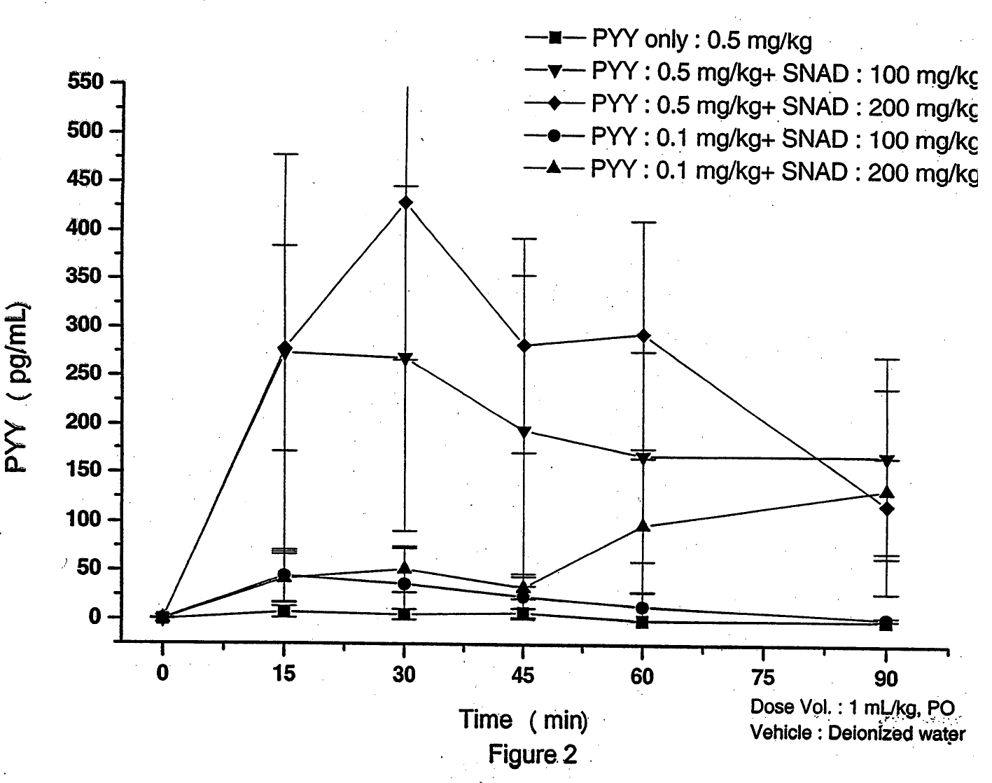

Blood samples were collected serially from the tail artery, or by cardiac puncture, typically at time=0, 15, 30, 45, 60 and 90 minutes. Serum PYY concentrations were quantified using a PYY[3-36] radioimmunoassay (Catalog #RK-059-02 from Phoenix Pharmaceuticals, Inc., Belmont, Calif.). Results from the animals in each group were averaged for each time point. The maximum of these averages (i.e., the mean peak serum PYY[3-36] concentration±standard deviation (SD)) is reported below in Table 1. No significant PYY[3-36] was detected in blood when the animals were dosed orally with PYY[3-36] alone. The results with the delivery agent SNAC and SNAD (±standard error) are also shown in FIGS. 1 and 2, respectively.

TABLE 1
Oral Delivery of PYY[3-36] in Rats

| | Delivery Agent | PYY[3-36] | volume | Mean Peak |
|---|---|---|---|---|
| Delivery | Dose | Dose | dose | Serum [PYY(3-36)] |
| Agent | (mg/kg) | (mg/kg) | (ml/kg) | (pg/ml) ± SD |
| 1 | 100 | 0.5 | 1 | 235.75 ± 181.7 |
| 1 | 200 | 0.5 | 1 | 442.098 ± 78.4 |
| 1 | 100 | 0.1 | 1 | 233.03 ± 127.5 |
| 1 | 200 | 0.1 | 1 | 313.14 ± 151.8 |
| 2 | 100 | 0.5 | 1 | 273.69 ± 457.5 |
| 2 | 200 | 0.5 | 1 | 430.29 ± 364.6 |
| 2 | 100 | 0.1 | 1 | 44.63 ± 53.53 |
| 2 | 200 | 0.1 | 1 | 134.37 ± 236.708 |
| 3 (none) | 0 | 0.5 | 1 | 8.8925 ± 8.290 |
| 4 | 200 | 0.3 | 1 | 830.246 ± 71.382 |
| 5 | 200 | 0.3 | 1 | 800.318 ± 40.61 |
| 6 | 200 | 0.3 | 1 | 812.946 ± 53.616 |
| 7 | 200 | 0.3 | 1 | 663.472 ± 135.394 |
| 8 | 200 | 0.3 | 1 | 276.82 ± 425.45 |
| 9 | 200 | 0.3 | 1 | 732.912 ± 266.578 |
| 10 | 200 | 0.3 | 1 | 846.416 ± 55.993 |
| 11 | 200 | 0.3 | 1 | 871.484 ± 119.461 |
| 12 | 200 | 0.3 | 1 | 845.682 ± 69.846 |
| 12 | 200 | 0.3 | 1 | 1047.46 ± 36.93 |
| 13 | 200 | 0.3 | 1 | 826.48 ± 379.12 |
| 14 | 200 | 0.3 | 1 | 913.536 ± 33.688 |
| 14 | 200 | 0.3 | 1 | 1083.38 ± 78.878 |
| 15 | 200 | 0.3 | 1 | 705.106 ± 75.906 |

Delivery Agent 1 is the monosodium salt of N-(8-[2-hydroxybenzoyl]-amino)caprylic acid (SNAC).

Delivery Agent 2 is the monosodium salt of N-(10-[2-hydroxybenzoyl]-amino)decanoic acid (SNAD).

Delivery Agent 4 is the monosodium salt of 8-(2-hydroxy-4-methoxybenzoylamino)octanoic acid.

Delivery Agent 5 is the monosodium salt of 8-(2,6-dihydroxybenzoylamino)octanoic acid.

Delivery Agent 6 is the monosodium salt of 8-(2-hydroxy-5-bromobenzoylamino)octanoic acid.

Delivery Agent 7 is the monosodium salt 8-(2-hydroxy-5-chlorobenzoylamino)octanoic acid.

Delivery Agent 8 is the monosodium salt of 8-(2-hydroxy-5-iodobenzoylamino)octanoic acid.

Delivery Agent 9 is the monosodium salt of 8-(2-hydroxy-5-methylbenzoylamino)octanoic acid.

Delivery Agent 10 is the monosodium salt of 8-(2-hydroxy-5-fluorobenzoylamino)octanoic acid.

Delivery Agent 11 is the monosodium salt of 8-(2-hydroxy-5-methoxybenzoylatnino)octanoic acid.

Delivery Agent 12 is the monosodium salt of 8-(3-hydroxyphenoxy)octanoic acid.

Delivery Agent 13 is the monosodium salt of 8-(4-hydroxyphenoxy)octanoic acid.

Delivery Agent 14 is the monosodium salt of 6-(2-cyanophenoxy)hexanoic acid.

Delivery Agent 15 is the monosodium salt of 8-(2-Hydroxyphenoxy)octyl-diethanolamine.

### Example 2

### Intraperitoneal Delivery of Peptide YY 13-36] in Rats

Intraperitoneal dosing solutions of PYY[3-36] were prepared in sterile saline solution (0.9% sodium chloride) at pH 7.5. The typical dosing and sampling protocols were as follows. Male Sprague-Dawley rats weighing between 240-320 g were fasted up to a maximum 24 hours before the experiments and administered ketamine (44 mg/kg) and thorazine (1.5 mg/kg) by intramuscular injection before the test article administration. Afterwards, the anesthetized animals were administered the test article by intraperitoneal injection. A dosing group of five animals was administered one of the dosing solutions.

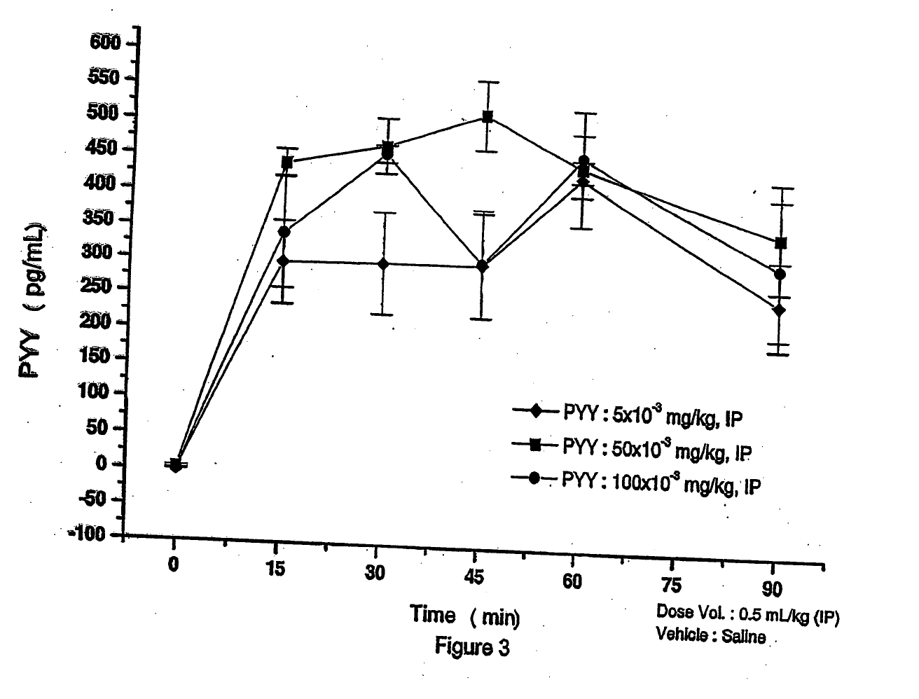

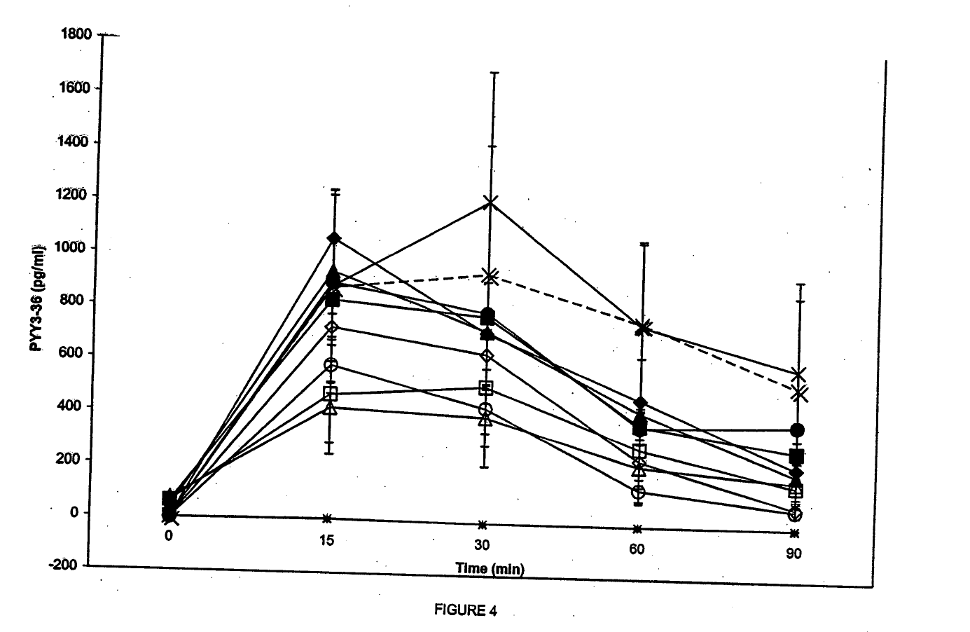

Blood samples were collected serially from the tail artery, or by cardiac puncture, typically at time=0, 15, 30, 45, 60 and 90 minutes. Serum PYY concentrations were quantified using a PYY[3-36] radioimmunoassay (Catalog #RK-059-02 from Phoenix Pharmaceuticals, Inc., Belmont, Calif.). Results from the animals in each group were averaged for each time point. The maximum of these averages (i.e., the mean peak serum PYY concentration) is reported below in Table 2. The results (i standard error) are also shown in FIG. 3.

TABLE 2
Intraperitoneal (IP) Delivery of PYY[3-36] in Rats

| | | | |
|---|---|---|---|
| | PYY[3-36] | Volume | Mean Peak |
| Method of | Dose | Dose | Serum [PYY] |
| Administration | (mg/kg) | (ml/kg) | (pg/ml) ± SD |

| | | | |
|---|---|---|---|
| IP | 0.005 | 0.5 | 435.19 ± 56.07 |
| IP | 0.05 | 0.5 | 521.02 ± 111.54 |
| IP | 0.1 | 0.5 | 464.48 ± 77.48 |

### Example 3

### Solid Oral Delivery of PYY[3-36] in Rats

### Example 3a

### Administration of Solid PYY 3-36 to Feed Restricted Rats

PYY[3-36] stock solution (80 mg/ml) prepared with deionized water was used.

About 0.08 mg/tablet (about 0.3 mg/kg) of PYY (about 1 μl) was added and blended with either about 13.5 or about 27 mg/tablet (about 50 or 100 mg/kg) Delivery Agent. Upper punch, lower punch and die of Carver 4350 manual pellet press with a Caplet shape model sold by Natoli Engineering Company, Inc. were treated with magnesium stearate (0.1%). About 13.58 or about 27.08 mg of mixed powder was fed into the die and a mini bead shape tablet was made at about 1000 PSI bar pressure. The resulting solid dosage form is about the size of a standard capsule size 9 (about 2.65 mm diameter and about 8.40 mm length) for the 27.08 mg size and about 2.65 mm diameter and about 4.20 mm length for the 13.58 mg solid.

Male Sprague Dawley rats (about 260 about 280 g) were fasted overnight and then anesthesized by standard CO₂ inhalation technique for about 10 to 30 seconds resulting in an anesthesized state for about less then one minute, preferably about 10 to about 30 seconds.

An oral dosing tube was used. The dosing tube was inserted into the rat's mouth and carefully threaded down the rats pharynx and esophagus about 8 cm to about 15 cm depending on the weight of the rat (typically about 11 cm). The solid dosage form was delivered into the distal esophagus and/or stomach by pressing the plunger of the oral dosing tube.

Blood samples were collected serially from the tail artery, by cardiac puncture, or as in this case by retro-orbitally, typically at time=0, 15, 30, 60 and 90 minutes. Serum PYY concentrations were quantified using a PYY[3-36] radioimmunoassay (Catalog #RK-059-02 from Phoenix Pharmaceuticals, Inc., Belmont, Calif.). Results from the animals in each group were averaged for each time point. The maximum of these averages (i.e., the mean peak serum PYY[3-36] concentration i standard deviation (SD)) is reported below in Table 3.

TABLE 3
Oral Delivery of PYY[3-36] in Feed Restricted Rats

| | | Delivery | | |
|---|---|---|---|---|
| | Method of | Agent dose | PYY[3-36] | Mean serum peak |
| Delivery Agent | Administration | (mg/kg) | dose (mg/kg) | of PYY (pg/ml) ± SD |
| 1 | Oral, solid dose, | 100 | 0.3 | 830.24 ± 341.32 |
| | 1 tablet per animal | | | |
| 1 | Oral, solid dose, | 50 | 0.3 | 511.5 ± 493.5 |
| | 1 tablet per animal | | | |
| 2 | Oral, solid dose, | 100 | 0.3 | 512.4 ± 484.2 |
| | 1 tablet per animal | | | |
| 2 | Oral, solid dose, | 50 | 0.3 | 536.3 ± 424.7 |
| | 1 tablet per animal | | | |
| 7 | Oral, solid dose, | 100 | 0.3 | 1064.18 ± 363.8 |
| | 1 tablet per animal | | | |
| 7 | Oral, solid dose, | 50 | 0.3 | 725.96 ± 110.78 |
| | 1 tablet per animal | | | |
| 7 | Oral, solid dose, | 100 | 0 | 14.35 ± 19.71 |
| | 1 tablet per animal | | | |
| 16 | Oral, solid dose, | 100 | 0.3 | 1294.2 ± 351.4 |
| | 1 tablet per animal | | | |
| 16 | Oral, solid dose, | 100 | 0.3 | 1560 ± 883.4 |
| | 1 tablet per animal | | | |
| 16 | Oral, solid dose, | 100 | 0.3 | 980.7 ± 49.7 |
| | 1 tablet per animal | | | |
| 16 | Oral, solid dose, | 50 | 0.3 | 617.4 ± 272.1 |
| | 1 tablet per animal | | | |
| 16 | Oral, solid dose, | 50 | 0.3 | 988.7 ± 288.2 |
| | 1 tablet per animal | | | |
| 16 | Oral, solid dose, | 50 | 0.3 | 847.3 ± 152 |
| | 1 tablet per animal | | | |
| 16 | Oral, solid dose, | 75 | 0.3 | 754.7 ± 539.8 |
| | 1 tablet per animal | | | |
| 16 | Oral, solid dose, | 25 | 0.1 | 274.1 ± 118.6 |
| | 1 tablet per animal | | | |
| 16 | Oral, solid dose, | 50 | 0.1 | 249.3 ± 144.0 |
| | 1 tablet per animal | | | |
| 16 | Oral, solid dose, | 25 | 0.3 | 953.8 ± 660.3 |
| | 1 tablet per animal | | | |
| 16 | Oral, solid dose, | 25 | 0.5 | 494.6 ± 318.2 |
| | 1 tablet per animal | | | |
| 16 | Oral, solid dose, | 50 | 0.5 | 715 ± 208.0 |
| | 1 tablet per animal | | | |
| 16 | Oral, solid dose, | 100 | 0.5 | 852.2 ± 1119 |
| | 1 tablet per animal | | | |
| 16 | Oral, solid dose, | 50 | 1 | 739.6 ± 409.6 |
| | 1 tablet per animal | | | |
| 2 and 16 | Oral, solid dose, | 25 and 25 | 0.3 | 791.22 ± 453.4 |
| | 1 tablet per animal | | | |
| 2 and 16 | Oral, solid dose, | 50 and 50 | 0.3 | 644.18 ± 595.4 |
| | 1 tablet per animal | | | |
| 16 | Oral, solid dose, | 50 | 0.3 | 421.08 ± 389.2 |
| | 1 tablet per animal | | | |
| 16 | Oral, solid dose, | 100 | 0.3 | 939.88 ± 166.61 |
| | 1 tablet per animal | | | |
| 17 | Oral, solid dose, | 100 | 0.3 | 897.18 ± 549.5 |
| | 1 tablet per animal | | | |
| 17 | Oral, solid dose, | 50 | 0.3 | 581.62 ± 236.94 |
| | 1 tablet per animal | | | |
| 18 | Oral, solid dose, | 100 | 0.3 | 1216.13 ± 661.31 |
| | 1 tablet per animal | | | |
| 18 | Oral, solid dose, | 50 | 0.3 | 940.33 ± 1090.65 |
| | 1 tablet per animal | | | |
| Control | Oral, solid dose, | 100 mg/kg of | 0.3 | 5.55 ± 12.40 |
| | 1 tablet per animal | mannitol. No | | |
| | | delivery | | |
| | | agent. | | |
| 19 | Oral, solid dose, | 100 | 0.3 | 728.4 ± 364.4 |
| | 1 tablet per animal | | | |
| 4 | Oral, solid dose, | 100 | 0.3 | 1821.82 ± 1513.84 |
| | 1 tablet per animal | | | |
| 9 | Oral, solid dose, | 100 | 0.3 | 1309.8 ± 836.33 |
| | 1 tablet per animal | | | |
| 10 | Oral, solid dose, | 50 | 0.3 | 897.18 ± 549.5 |
| | 1 tablet per animal | | | |
| 20 | Oral, solid dose, | 100 | 0.3 | 695.8 ± 274.3 |
| | 1 tablet per animal | | | |
| 11 | Oral, solid dose, | 100 | 0.3 | 2330.63 ± 963.7 |
| | 1 tablet per animal | | | |

Delivery Agent 16 is the disodium salt of N-(10-[2-hydroxybenzoyl]-amino)decanoic acid (SNAD).

Delivery Agent 17 is the disodium salt of 8-(4-hydroxyphenoxy)octanoate.

Delivery Agent 18 is the monosodium salt of 8-(4-hydroxyphenoxy)octanoate.

Delivery Agent 19 is the disodium salt of 8-(2-hydroxy-4-methoxybenzoylamino)octanoic acid.

Delivery Agent 20 is the disodium salt of8-(2-hydroxy-5-methoxybenzoylamino)octanoic acid.

Mannitol was used as a control

### Example 3b

### Administration of Solid PYY[3-36] to Non-feed Restricted Rats

Rats were subjected to solid oral dosage forms consisting of PYY [3-36] and a carrier agent as described above in Example 3a, except no food restriction was imposed upon the rats prior to administration of the solid dosage forms. Serum concentrations of PYY were determined for each of the animals at various time points and the concentration from the animals in each group were averaged for each time point. The maximum of these averages (i.e., the mean peak serum PYY[3-36] concentration±standard deviation (SD)) is reported below in Table 4.

TABLE 4
Oral Delivery of PYY[3-36] in Non-Feed Restricted Rats

| | | Delivery | | |
|---|---|---|---|---|
| | Method of | Agent dose | PYY[3-36] | Mean serum peak |
| Delivery Agent | Administration | (mg/kg) | dose (mg/kg) | of PYY (pg/ml) ± SD |
| 16 | Oral, solid dose, | 50 | 0.3 | 1101 ± 1197 |
| | 1 tablet per animal | | | |
| 16 | Oral, solid dose, | 100 | 0.3 | 1011.5 ± 1287 |
| | 1 tablet per animal | | | |
| 16 | Oral, solid dose, | 100 | 0.5 | 1735.6 ± 1108 |
| | 1 tablet per animal | | | |

### Example 3c

### Administration of Mini-tablets to Feed Restricted Rats

Male Sprague Dawley rats were fasted overnight. Mini-tablets (about 2.5 mm diameter) containing PYY[3-36] and SNAD were prepared by physical blend and compression and administered to the rats by oral gavage. Serum PYY[3-36] levels were determined by radioimmunoassay. Fifteen minutes after the administration of one tablet containing 0.3 and 100 mg per kg (body weight) of PYY[3-36] and SNAD, respectively, serum PYY[3-36] increased to 940±74 pg/ml (n=5, mean±s.e.m.) and remained above basal levels for up to 90 minutes. No changes in serum PYY[3-36] were detected when either PYY[3-36] or SNAD were administered alone.

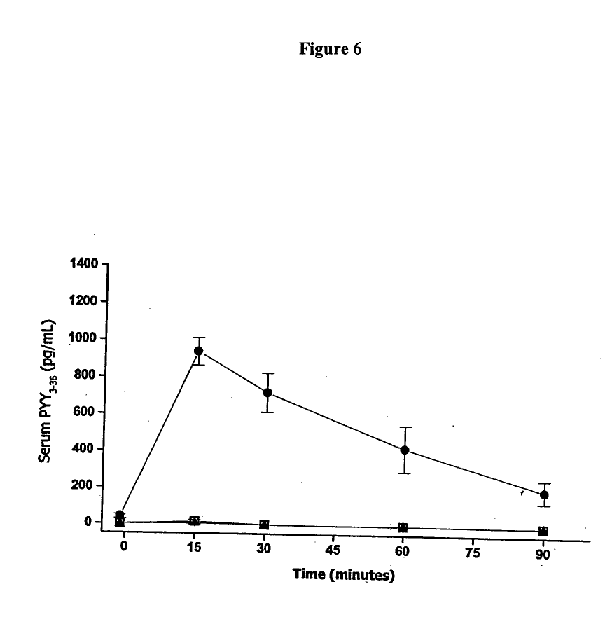

The results over the 90 minute test interval are shown in FIG. 6.

### Example 4

### Peptide YY (PYY[3-36]) Solid Oral Delivery in Rhesus Monkeys

About 1 mg/tablet of PYY solid powder was gradually added and blended with either 50 or 100 mg/tablet Delivery Agent. Upper punch, lower punch and die of Carver 4350 manual pellet press with a Caplet shape model sold by Natoli Engineering Company, Inc. were treated with magnesium stearate (0.1%). About 51 or about 101 mg of mixed powder was fed into the die and a mini bead shape tablet was made at about 1000 PSI bar pressure. The resulting solid dosage form is about 3 mm diameter and about 1 mm in height for the 51 mg solid or about 3 mm diameter and about 2 mm in height for the 101 mg solid.

For each dosage level, 2 male and 2 female Rhesus Monkeys weighing between 3.5-5.0 kg were fasted up overnight before the experiments and food was returned about 2 hours after dosing of the solid. Water was withheld from 30 minutes prior to dosing until 30 minutes after dosing, except for those quantities used for dosing.

Each solid dosage form was delivered to the rear of the mouth using a pill gun. After release of the solid dosage form, 5 ml of reverse osmosis water was administered into the oral cavity to facilitate swallowing. Following delivery, the oral cavity was inspected to ensure that the solid was swallowed.

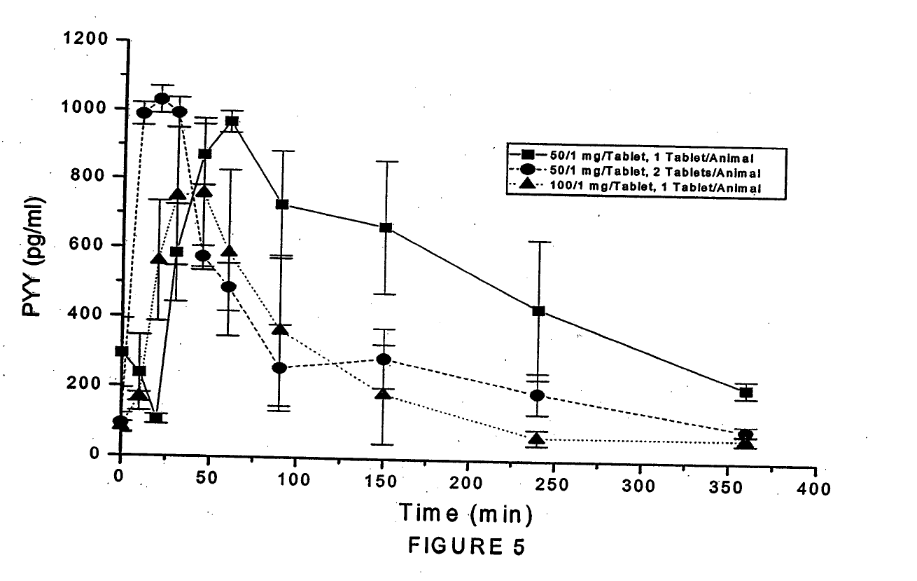

Blood samples (about 1.3 ml) were collected serially from the femoral, brachial or saphenous vein, typically at time=0 (predose), 10, 20, 30, 45, 60, 90, 150, 240 and 360 minutes post dose. The samples were placed in serum separating tubes and left at room temperature for 30-45 minutes to allow clotting. The samples were then centrifuged at about 2-8° C. for 10 minutes at 2500 rpm. The resulting serum was transferred into a tube and placed on dry ice and then stored frozen at −70±10° C. until assayed. Serum PYY concentrations were quantified using a PYY[3-36] radioimmunoassay (Catalog #RK-059-02 from Phoenix Pharmaceuticals, Inc., Belmont, Calif.). Results from the animals in each group were averaged for each time point. The maximum of these averages (i.e., the mean peak serum PYY[3-36] concentration±standard deviation (SD)) is reported below in Table 4. The results (±standard error) are also shown in FIG. 5.

TABLE 4
Oral Delivery of PYY[3-36] in Rhesus Monkeys

| | | | | Dose per animal | Mean |
|---|---|---|---|---|---|
| | | Delivery | PYY[3-36] | of: Delivery | serum peak |
| Delivery | Method of | Agent dose | dose | Agent (mg)/ | of PYY |
| Agent | Administration | (mg/tablet) | (mg/tablet) | PYY[3-36] (mg) | (pg/ml) ± SD |
| 1 | Oral, solid dose, 1 | 50 | 1 | 50/1 | 969 ± 60.72 |
| | tablet per animal | | | | |
| 1 | Oral, solid dose, 2 | 50 | 1 | 100/2 | 1030.9 ± 76.88 |
| | tablet per animal | | | | |
| 1 | Oral, solid dose, 1 | 100 | 1 | 100/1 | 757.8 ± 441.02 |
| | tablet per animal | | | | |

Delivery Agent 1 is the monosodium salt of N-(8-[2-hydroxybenzoyl]-amino)caprylic acid (SNAC).

### Example 5

### Effect of Orally Administered PYY[3-36] on 4-hour Food Intake

A PYY[3-36] stock solution (80 mg/ml) prepared with deionized water was used to prepare PYY[3-36] tablets.

About 0.132 mg/tablet (about 0.5 mg/kg) of PYY[3-36] (about 1.7 μl) was added and blended with about 27 mg/tablet (100 mg/kg) delivery agent SNAD. Upper punch, lower punch and die of a Carver 4350 manual pellet press with a Caplet shape model (available from Natoli Engineering Company, Inc.) were treated with magnesium stearate (0.1%). 27.132 mg of mixed PYY[3-36]/SNAD powder was fed into the die and a mini bead shape tablet was made at about 1000 PSI bar pressure. The resulting solid dosage form was about the size of a standard capsule size 9 (about 2.65 mm diameter and about 8.40 mm length).

Placebo tablets contained only SNAD, about 27 mg/tablet (100 mg/kg), and were prepared in the same way.

Male Sprague Dawley rats (about 260 about 280 g) were fasted for 24 hours prior to dosing. Each rat was dosed with either one PYY[3-36]/SNAD tablet or one placebo tablet.

An oral dosing tube was used. The dosing tube was inserted into the rat's mouth and carefully threaded down the rats pharynx and esophagus about 8 cm to about 15 cm depending on the weight of the rat (typically about 11 cm). The solid dosage form was delivered into the distal esophagus and/or stomach by pressing the plunger of the oral dosing tube. No anesthesia was used for dosing.

Food was weighed and given to the rats one hour post-dose. Four hours later, the food was removed and weighed. The amount of food consumed was determined from the food weight difference.

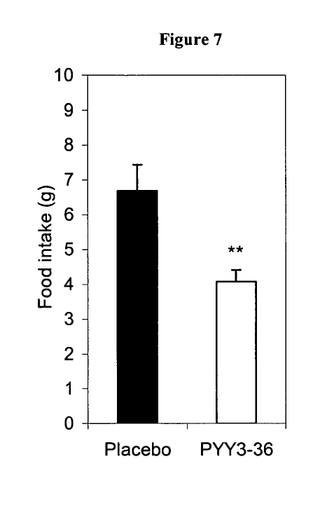

4-hour cumulative food intake in grams (mean±s.e.m.) are shown in Table 5 and FIG. 7. Double asterisk denotes P<0.01.

TABLE 5
4-Hour Food Intake

| | 4-hour food | Standard | |
|---|---|---|---|
| Group | intake (g) | Error | n |
| PYY[3-36]/ | 4.09 | 0.33 | 9 |
| SNAD | | | |
| Placebo | 6.69 | 0.76 | 9 |

As shown above and in FIG. 7, rats receiving one PYY[3-36] tablet consumed significantly less food than those receiving one placebo tablet.

### Example 6

### Effect of Orally Administered PYY[3-36] on Weight Gain and Food Intake After a 7-day Treatment

A PYY[3-36] stock solution (110 mg/ml) prepared with deionized water was used to prepare PYY[3-36] tablets.

Mini bead shape tablets containing about 0.11 mg/tablet (about 0.5 mg/kg) of PYY[3-36] and about 16.5 mg/tablet (75 mg/kg) delivery agent SNAD were prepared by the procedure described in Example 6.

Placebo tablets contained only SNAD, about 16.5 mg/tablet (75 mg/kg), were also prepared as described by the procedure described in Example 6.

Male Sprague Dawley rats (about 220 g) were dosed twice daily for 7 days with either one PYY[3-36] tablet or a placebo tablet. Prior to dosing, the rats were anesthesized by standard CO₂ inhalation technique for about 10 to 30 seconds resulting in an anesthesized state for about less then one minute, typically about 10 to about 30 seconds. Food was removed prior to dosing and returned 30 minutes after dosing.

An oral dosing tube was used. The dosing tube was inserted into the rat's mouth and carefully threaded down the rats pharynx and esophagus about 8 cm to about 15 cm depending on the weight of the rat (typically about 11 cm). The solid dosage form was delivered into the distal esophagus and/or stomach by pressing the plunger of the oral dosing tube.

Food consumption was determined every 24 hours from the weight difference between the food given to and food left over by each rat. The body weight of each rat was determined every 24 hours.

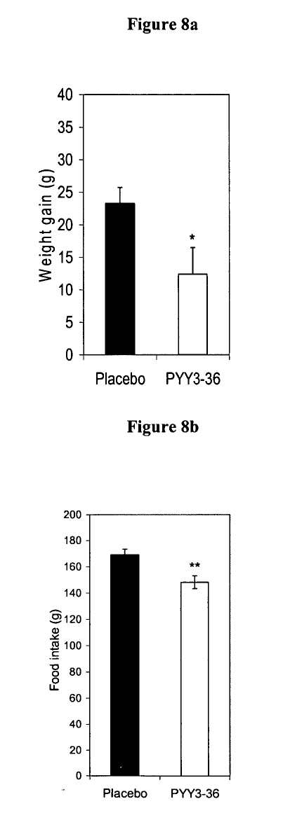

The results are shown in Table 6 and FIGS. 8a and 8b. Weight gain in grams during a 7-day treatment is shown in FIG. 8a. Asterisk denotes P<0.05. Cumulative food intake in grams during a 7-day treatment (mean±s.e.m.) is shown in FIG. 8b. Double asterisk denotes P<0.01.

TABLE 6
Weight Gain During Seven-Day Treatment

| | Weight gain | | Food intake | | |
|---|---|---|---|---|---|
| | after 7-day | Standard | during 7-day | Standard | |
| Group | treatment(g) | Error | treatment(g) | Error | n |
| PYY[3-36]/ | 12 | 4 | 148 | 5 | 7 |
| SNAD | | | | | |
| Placebo | 23 | 2 | 169 | 4 | 7 |

As shown above and in FIGS. 8a and 8b, rats receiving PYY[3-36]/SNAD gained significantly less weight and consumed less food than those receiving placebo. PYY[3-36] had no effect on gastric emptying. The amount of food found in the stomachs at the end of the 7-day study was minimal and comparable for placebo and treated animals. No pathology or other findings were detected in the gastrointestinal tract during gross necropsies performed at the end of the study.

### Example 7

### Dosage Forms of Disodium Salt of SNAD and PYY [3-36] and Intraperitoneal dosing of PYY [3-36]

Liquid dosage forms were prepared as follows. PYY[3-36] stock solution (80 mg/ml) prepared with deionized water. A liquid solution of delivery agent was prepared by dissolving the disodium salt of SNAD in water. The pH of the resulting disodium SNAD solution was about 10. Aliquots of the disodium SNAD solution were mixed with aliquots of PYY[3-36] solution, which had a pH of about 8. Liquid dosage forms having between about 100 and 200 mg/kg disodium SNAD and between about 0.3 and 1 mg/kg PYY[3-36] were prepared according to this procedure. The final pH of the liquid dosage forms was between about 9 and 10.

Solid dosage forms were prepared as follows. About 0.08 mg/tablet (about 0.3 mg/kg) of PYY (about 1 μl) was added and blended with either about 13.5 or about 27 mg/tablet (about 50 or 100 mg/kg) Delivery Agent. Upper punch, lower punch and die of Carver 4350 manual pellet press with a Caplet shape model sold by Natoli Engineering Company, Inc. were treated with magnesium stearate (0.1%). About 13.58 or about 27.08 mg of mixed powder was fed into the die and a mini bead shape tablet was made at about 1000 PSI bar pressure. The resulting solid dosage form is about the size of a standard capsule size 9 (about 2.65 mm diameter and about 8.40 mm length) for the 27.08 mg size and about 2.65 mm diameter and about 4.20 mm length for the 13.58 mg solid.

Male Sprague Dawley rats (about 260 about 280 g) were fasted overnight and then anesthesized by standard CO₂ inhalation technique for about 10 to 30 seconds resulting in an anesthesized state for about less then one minute, preferably about 10 to about 30 seconds.

An oral dosing tube was used. The dosing tube was inserted into the rat's mouth and carefully threaded down the rats pharynx and esophagus about 8 cm to about 15 cm depending on the weight of the rat (typically about 11 cm). The solid dosage form was delivered into the distal esophagus and/or stomach by pressing the plunger of the oral dosing tube.

Blood samples were collected serially from the tail artery, by cardiac puncture, or as in this case by retro-orbitally, typically at time=0, 15, 30, 60 and 90 minutes. Serum PYY concentrations were quantified using a PYY[3-36] radioimmunoassay (Catalog #RK-059-02 from Phoenix Pharmaceuticals, Inc., Belmont, Calif.). Results from the animals in each group were averaged for each time point. The maximum of these averages (i.e., the mean peak serum PYY concentration) is reported below in Table 7.

TABLE 7
Delivery of PYY[3-36] in Rats

| | Disodium | PYY(3-36) | |
|---|---|---|---|
| Method of | SNAD dose | dose | Mean serum peak of |
| Administration | (mg/tablet) | (mg/tablet) | PYY (pg/ml) ± SD |
| Oral, solid dose, | 100 | 0.5 | 1518.36 ± 464.98 |
| 1 tablet per animal | | | |
| Oral, solid dose, | 100 | 0.5 | 3685.8 ± 880.43 |
| 1 tablet per animal | | | |
| Oral, liquid dose | 150 | 0.5 | 2215.6 ± 1543.01 |
| Oral, liquid dose | 150 | 1 | 4697.8 ± 3009.48 |
| Oral, liquid dose | 150 | 1 | 2432.4 ± 1702.55 |
| Oral, liquid dose | 100 | 0.3 | 1008.2 ± 145.16 |
| Oral, liquid dose | 200 | 0.3 | 805.67 ± 583.78 |
| Oral, liquid dose | 200 | 0 | 0 |
| Intraperitoneal | 0 | 0.05 | 3671 ± 244.7 |
| injection | | | |
| Intraperitoneal | 0 | 0.05 | 6292.8 ± 1365.1 |
| injection | | | |

### Example 8

### Peptide YY (PYY 13-36]). Solid Oral Delivery in Cynomolgus Monkeys

Capsules were prepared as follows. About 1 mg of PYY solid powder was gradually added and blended with either 50 or 100 mg of SNAD. Pre-weighed size 2 capsules were packed with the blend using a clean metal spatula. The final capsules were reweighed and determined to contain >98% of the transferred blend.

Tablets were prepared as follows. About 1 mg/tablet of PYY solid powder was gradually added and blended with about 50 mg/tablet Delivery Agent. Upper punch, lower punch and die of Carver 4350 manual pellet press with a Caplet shape model sold by Natoli Engineering Company, Inc. were treated with magnesium stearate (0.1 %). About 51 mg of mixed powder was fed into the die and a mini bead shape tablet was made at about 1000 PSI bar pressure. The resulting solid dosage form is about 3 mm diameter and about 1 mm in height.

For each dosage level, 2 male and 2 female Cynomolgus Monkeys weighing between 3.5 and 5.0 kg were fasted up overnight before the experiments and food was returned about 2 hours after dosing of the solid. Water was withheld from 30 minutes prior to dosing until 30 minutes after dosing, except for those quantities used for dosing.

Each solid dosage form was delivered directly into the stomach using a gavage tube. The capsule or tablet was ejected by air flush.

Blood samples (about 1.3 ml) were collected serially from the femoral, brachial or saphenous vein, typically at time=0 (predose), 10, 20, 30, 45, 60, 90, 150, 240 and 360 minutes post dose. The samples were placed in serum separating tubes and left at room temperature for 30-45 minutes to allow clotting. The samples were then centrifuged at about 2-8° C. for 10 minutes at 2500 rpm. The resulting serum was transferred into a tube and placed on dry ice and then stored frozen at −70±10° C. until assayed. Serum PYY concentrations were quantified using a PYY[3-36] radioimmunoassay (Catalog #RK-059-02 from Phoenix Pharmaceuticals, Inc., Belmont, Calif.). Results from the animals in each group were averaged for each time point. The maximum of these averages (i.e., the mean peak serum PYY concentration) is reported below in Table 8.

TABLE 8
Results of PYY [3-36] Solid Oral Delivery in
Cynomolgus monkeys

| | | Com- | | |
|---|---|---|---|---|
| | | pound | | |
| | | dose | PYY(3-36) | Mean serum |
| Com- | Method of | (mg/ | dose | peak of PYY |
| pound | Administration | tablet) | (mg/tablet) | (pg/ml) ± SD |
| SNAD | Oral, solid dose, | 50 | 1 | 419.68 ± 320.44 |
| | 1 capsule per animal | | | |
| SNAD | Oral, solid dose, | 100 | 1 | 432.02 ± 309.47 |
| | 1 capsule per animal | | | |
| SNAD | Oral, solid dose, | 50 | 1 | 925.86 ± 794.03 |
| | 1 tablet per animal | | | |

The above-mentioned patents, applications, test methods, and publications are hereby incorporated by reference in their entirety.

Many variations of the present invention will suggest themselves to those skilled in the art in light of the above detailed description. All such obvious variations are within the fully intended scope of the appended claims.

1. A pharmaceutical composition comprising (a) peptide YY, a peptide YY agonist, or a mixture thereof and (b) a delivery agent of the formula 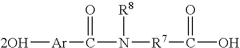
or a salt thereof, wherein:
Ar is phenyl or naphthyl;
Ar is optionally substituted with one or more of —OH, halogen, C₁-C₄ alkyl, C₁-C₄ alkenyl, C₁-C₄ alkoxy or C₁-C₄ haloalkoxy;
R⁷ is selected from C₄-C₂₀ alkyl, C₄-C₂₀ alkenyl, phenyl, naphtnyl, (C₁-C₁₀ alkyl) phenyl, (C₁-C₁₀ alkenyl)phenyl, (C₁-C₁₀ alkyl) naphthyl, (C₁-C₁₀ alkenyl) naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);
R⁸ is selected from hydrogen, C₁ to C₄ alkyl, C₂ to C₄ alkenyl, C₁ to C₄ alkoxy, and C₁-C₄ haloalkoxy;
R⁷is optionally substituted with C₁ to C₄ alkyl, C₂ to C₄ alkenyl, C₁ to C₄ alkoxy, C₁-C₄ haloalkoxy, —OH, —SH, —CO₂R⁹, or any combination thereof;
R⁹ is hydrogen, C₁ to C₄ alkyl, or C₂ to C₄ alkenyl; and
R⁷ is optionally interrupted by oxygen, nitrogen, sulfur or any combination thereof; with the proviso that the compounds are not substituted with an amino group in the position alpha to the acid group.

2. A pharmaceutical composition comprising (a) peptide YY, a peptide YY agonist, or a mixture thereof and (b) a delivery agent of the formula 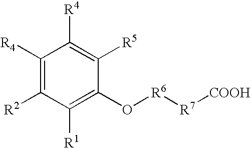
or a salt thereof, wherein
R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹ (R¹²)⁻;
R⁵ is H, —OH, —NO₂, halogen, —CF₃, —NR¹⁴R¹⁵, —N⁺R¹⁴R¹⁵R¹⁶ (R¹³)⁻, amide, C₁-C₁₂ alkoxy, C₁-C₁₂ alkyl, C₂-C₁₂ alkenyl, carbarmrate, carbonate, urea, or —C(O)R¹⁸;
R⁵ is optionally substituted with halogen, —OH, —SH, or —COOH;
R⁵ is optionally interrupted by O, N, S, or —C(O)—;
R⁶ is a C₁-C₁₂ alkylene, C₂-C₁₂ alkenylene, or arylene;
R⁶ is optionally substituted with a C₁-C₄ alkyl, C₂-C₄ alkenyl, C₁-C₄ alkoxy, —OH, —SH, halogen, —NH₂, or —CO₂R⁸;
R⁶ is optionally interrupted by O or N;
R⁷ is a bond or arylene;
R⁷ is optionally substituted with —OH, halogen, —C(O)CH₃, —NR¹⁰R¹¹, or —N⁺R¹⁰R¹¹R¹² (R¹³)⁻;
each occurrence of R⁸ is independently H, C₁-C₄ alkyl, C₂-C₄ alkenyl, or —NH₂;
R⁹, R¹⁰, R¹¹, and R¹² are independently H or C₁-C₁₀ alkyl;
R¹³ is a halide, hydroxide, sulfate, tetrafluoroborate, or phosphate; and
R¹⁴, R¹⁵ and R¹⁶ are independently H, C₁-C₁₀ alkyl, C₁-C₁₀ alkyl substituted with —COOH, C₂-C₁₂ alkenyl, C₂-C₁₂ alkenyl substituted with —COOH, or —C(O)R¹⁷;
R¹⁷ is —OH, C₁-C₁₀ alkyl, or C₂-C₁₂ alkenyl; and
R¹⁸ is H, C₁-C₆ alkyl, —OH, —NR¹⁴R¹⁵, or N⁺R¹⁴R¹⁵R¹⁶(R¹³).

3. A pharmaceutical composition comprising (a) peptide YY, a peptide YY agonist, or a mixture thereof and (b) a delivery agent of the formula 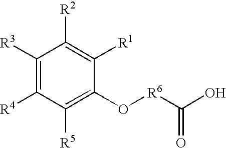
or a salt thereof, wherein
R¹, R², R³, R⁴ and R⁵ are independently H, —CN, —OH, —OCH₃, or halogen, at least one of R¹, R², R³, R⁴ and R⁵ being —CN; and
R⁶ is a C₁-C₁₂ linear or branched alkylene, alkenylene, arylene, alkyl(arylene) or aryl(alkylene).

4. A pharmaceutical composition of claim 1, wherein the delivery agent is selected from the group consisting of the monosodium salt of N-(8-[2-hydroxybenzoyl]-amino)caprylic acid, the monosodium salt of N-(10-[2-hydroxybenzoyl]-amino)decanoic acid, the monosodium salt of 8-(2-hydroxy-4-methoxybenzoylamino)octanoic acid, the monosodium salt of 8-(2,6-dihydroxybenzoylamino)octanoic acid, the monosodium salt of 8-(2-hydroxy-5-bromobenzoylamino)octanoic acid, the monosodium salt 8-(2-hydroxy-5-chlorobenzoylamino)octanoic acid, the monosodium salt of 8-(2-hydroxy-5-iodobenzoylamino)octanoic acid, the monosodium salt of 8-(2-hydroxy-5-methylbenzoylamino)octanoic acid, the monosodium salt of 8-(2-hydroxy-5-fluorobenzoylamino)octanoic acid, the monosodium salt of 8-(2-hydroxy-5-methoxybenzoylamino)octanoic acid, the monosodium salt of 8-(3-hydroxyphenoxy)octanoic acid, the monosodium salt of 8-(4-hydroxyphenoxy)octanoic acid, the monosodium salt of 6-(2-cyanophenoxy)hexanoic acid, the monosodium salt of 8-(2-Hydroxyphenoxy)octyl-diethanolamine, disodium salt of N-(10-[2-hydroxybenzoyl]-amino)decanoic acid, the disodium salt of 8-(4-hydroxyphenoxy)octanoate, the monosodium salt of 8-(4-hydroxyphenoxy)octanoate, the disodium salt of 8-(2-hydroxy-4-methoxybenzoylamino)octanoic acid, and the disodium salt of 8-(2-hydroxy-5-methoxybenzoylamino)octanoic acid.

5. The pharmaceutical composition of claim 1 wherein the delivery agent is N-(8-[2-hydroxybenzoyl]-amino)caprylic acid or a pharmaceutically acceptable salt thereof.

6. The pharmaceutical composition of claim 1 wherein the delivery agent is N-(10-[2-hydroxybenzoyl]-amino)decanoic acid or a pharmaceutically acceptable salt thereof.

7. The pharmaceutical composition of claim 1, wherein the peptide YY agonist is selected the functional domain of PYY, active fragments of PYY, derivatives of PYY, fragments of PYY, and analogs of PYY.

8. The pharmaceutical composition of claim 1, wherein the peptide YY agonist is PYY[3-36].

9. A dosage unit form comprising:
(A) the pharmaceutical composition of claim 1; and
(B) (a) an excipient,
(b) a diluent,
(c) a disintegrant,
(d) a lubricant,
(e) a plasticizer,
(f) a colorant,
(g) a dosing vehicle, or
(h) any combination thereof.

10. The dosage unit form of claim 9, wherein the dosage unit form is in the form of a tablet, a capsule, a particle, a powder, a sachet, or a liquid.

11. The dosage unit form of claim 9, wherein the dosing vehicle is a liquid selected from the group consisting of water, aqueous propylene glycol, phosphate buffer, 1,2-propane diol, ethanol, and any combination thereof.

12. A method for administering an effective amount of peptide YY or a peptide YY agonist to a patient in need of thereof, comprising the step of orally administering the pharmaceutical composition of claim 1.

13. A method of treating obesity in a patient in need thereof, comprising the step of administering to the patient an effective amount of the pharmaceutical composition of claim 1.

14. A method of treating a condition or disorder that may be alleviated by reducing nutrient availability in a patient in need thereof, comprising the step of administering an animal an effective amount of the pharmaceutical composition of claim 1.

15. The method of claim 14 wherein the condition or disorder is selected from the group consisting of hypertension, dyslipidemia, cardiovascular risk, an eating disorder, insulin-resistance, obesity and diabetes mellitus.

16. A method of reducing nutrient uptake in a patient in need thereof, comprising the step of administering an effective amount of the pharmaceutical composition of claim 1.

17. A method of improving the lipid profile in a patient in need thereof, comprising the step of administering an effective amount of the pharmaceutical composition of claim 1.

18. The method of claim 12 wherein the peptide YY agonist is PYY[3-36] and the delivery agent is N-(8-[2-hydroxybenzoyl]-amino)caprylic acid or N-(10-[2-hydroxybenzoyl]-amino)decanoic acid or a pharmaceutically acceptable salt thereof.

19. A method of improving the bioavailability of peptide YY or a peptide YY agonist in an animal, the method comprising the step of administering a formulation of claim 1.

20. A method of preparing a pharmaceutical composition comprising the step of mixing at least one of a peptide YY and a peptide YY agonist and a delivery agent of the formula 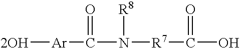
or a salt thereof, wherein:
Ar is phenyl or naphthyl:
Ar is optionally substituted with one or more of —OH, halogen, C₁-C₄ alkyl C₁-C₄ alkenyl, C₁-C₄ alkoxy or C₁-C₄ haloalkoxy:
R⁷ is selected from C₄-C₂₀ alkyl C₄-C₂₀ alkenyl, phenyl, naphthyl (C₁-C₁₀ alkyl) phenyl, (C₁-C₁₀ alkenyl)phenyl, (C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);
R⁸ is selected from hydrogen, C₁ to C₄ alkyl, C₁ to C₄ alkenyl, C₁ to C₄ alkoxy, and C₁-C₄ haloalkoxy;
R⁷ is optionally substituted with C₁ to C₄ alkyl, C₂ to C₄ alkenyl, C₁ to C₄ alkoxy, C₁-C₄ haloalkoxy, —OH, —SH, —CO₂R⁹, or any combination thereof;
R⁹ is hydrogen, C₁ to C₄ alkyl, or C₂ to C₄ alkenyl; and
R⁷ is optionally interrupted by oxygen, nitrogen, sulfur or any combination thereof, with the proviso that the compounds are not substituted with an amino group in the position alpha to the acid group.
